(19) United States
(12) Patent Application Publication
(10) Pub. No.: US 2005/0197665 A1
(43) Pub. Date: Sep. 8, 2005

(54) Tongue cleaners

(76) Inventors: Ralph A. Teed, Newark, AR (US); Don Hitt, Rogers, AR (US)

Correspondence Address: DENNIS B. HAASE, P.O. BOX 6206, HOT SPRINGS, AR, 71902-6206, US

(21) Appl. No.: 10/794,044

(22) Filed: Mar. 8, 2004

(51) Int. Cl. A61B 17/24

(52) U.S. Cl. 606/161

(57) ABSTRACT

A tongue cleaner having a cup and a handle connected to the cup. The cup having a leading edge, and a scraping edge inboard of the leading edge so positioned as to contact the surface of the tongue when the cleaner is moved from aft toward the front of the tongue, to thereby gently remove material from the tongue and retain such material in the cup.

The present invention relates generally to tongue cleaners and represents an improvement in such devices for use in scraping bacteria, or the like, from remote areas of the surface of one's tongue.

### BACKGROUND OF THE INVENTION

1. Field of the Invention

Halitosis is an ugly word. It is ugly to look at, and it even sounds ugly. Yet, the term (a more scientific term for what many simply refer to as bad breath) has a critical bearing on one's personal hygiene as well at the perception of others who come in contact with a person suffering from this rather common malady. Indeed, it has become so pervasive in some circles that it has its own phobia or syndrome, referred to as halitophobia.

While there is a popular belief that bad breath emanates from some gastrointestinal condition, research has pointed to bacteria in the posterior region of the tongue as a principal source of bad breath. The offending bacteria lives in the nooks and crannies of the tongue's surface, relatively undisturbed by oxygen and saliva.

The American Dental Association has been quoted as follows:
“ . . . halitosis experts agree that the dominant cause of Bad Breath is the bacteria on the tongue.
   The surface of the tongue is one of the main breeding grounds for the bacteria that generates foul smelling sulfides and fills the mouth with noxious gases that smell like rotten eggs and barnyard.”
      The ADA, not surprisingly, recommends a regimen of good oral hygiene, which includes removal of such bacteria, along with brushing and flossing, as a means of controlling, if not eliminating, bacteria which causes bad breath.

A surprisingly large industry has developed about the human desire to be free of bad breath. Gargles, pastes, rinses and mouth washes are legion; there are sprays, swabs, dental flosses and even pills that tout their ability to rid an individual of bad breath. There are also mechanical devices, such as brushes and scrapers, which seek to dislodge and wipe away offending bacteria.

It is an improved scraping device which is the focus of the present invention.

2. Overview of the Prior Art

Over the last several years, as the emphasis on oral hygiene became more prominent, there have been a number of developments among researchers and manufacturers of dental related products to design and market tongue scraping devices. Unfortunately, many of those efforts focused on “looking different” as distinguished from “performing better”, perhaps a testament to a society which responds more readily to looking good.

Early on, Mr. McNeill devised, and patented his cleaning device, which is chronicled in U.S. Pat. No. 2,491,274. McNeill, while not a pioneer in the area of tongue scrapers, was an early advocate of a sponge, fitted over a handle for removing material from the back of the tongue.

There is a relative dirth of patent activity after McNeill until the issuance of Gaskins Pat. No. DES. 291,001 which teaches a tongue scraper which appears to resemble a curved rasp, or file.

Next, Ly applied for and was issued DES. 360,262 which resembles a safety razor with a single hooked edge with a decorative design on the forward end.

Baker, in his design Pat. DES. 367,707, divines another scraper which includes what appears to be a razor type handle with a blade like insert facing downwardly.

Some twelve years after Gaskins, Welt et al. issued U.S. Pat. No. 5,984,935, which teaches a tongue cleaner having a series of upstanding blades aligned in spaced relation on a head formed with a handle. In the same general period, Patel filed for and was issued U.S. Pat. No. 5,779,475 for a closed loop tongue scraper.

Finally, Cho Pat. Des. 391,370 depicts a spoon like plastic tongue scraper having a severely canted handle attached to the spoon.

In addition to the patent art, there are a variety of tongue scrapers found on the Internet, all of which resemble, to a greater or lesser extent, those depicted in the patent art.

### SUMMARY OF THE INVENTION

The present invention, very simply, relates to an improved tongue scraper, having as its primary objective, providing an individual with a device which will comfortably reach to the rear of one's tongue and which is movable over the surface of the tongue to remove bacteria and other impediments thereon in an especially fast and efficient manner.

It is another object of the present invention to provide one concerned with oral hygiene with a device that will remove and collect bacteria and other accumulated residue from the tongue to thereby permit appropriate disposal of such material in a simple and sanitary manner.

Yet another objective of the present invention is to provide the market with an ergonomic design for a tongue scraper which fits the user's hand and is readily manipulated to accomplish the objectives attributable to the invention.

The foregoing, as well as other objects and advantages of the present invention, will become apparent from a reading of the following Detailed Description of a Preferred Embodiment, taken in conjunction with the drawings, wherein:

### DESCRIPTION OF THE DRAWINGS

FIG. 1 is a pictorial representation of a tongue scraper constructed in accordance with the present invention, depicted in perspective with its working side up in order to show certain features thereof;

FIG. 2 is side elevation of the tongue scraper of FIG. 1;

FIG. 3 is a top plan view of the tongue scraper of FIG. 1; and

FIG. 4 is a side elevation, partially sectioned, showing the tongue scraper of FIG. 1 as it would function in actual use as it moves across the rear or posterior surface of a representative tongue.

### DETAILED DESCRIPTION OF A PREFERRED EMBODIMENT

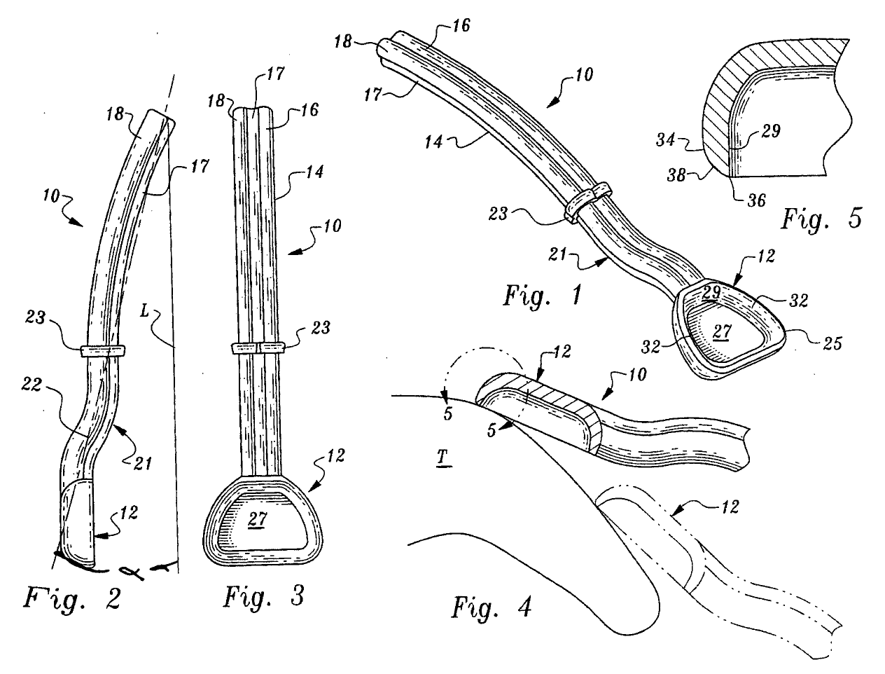

With reference now to the drawings, and initially to FIG. 1, a tongue cleaning device, often referred to as a tongue scraper, constructed in accordance with the present invention is depicted at 10.

The tongue scraper 10 includes a reservoir cup 12 which may be integrally formed with, or otherwise attached to, a handle 14. The handle, which permits digital manipulation of the cup, is formed with a plurality of longitudinally extending ribs, three of which, 16, 17 and 18 are shown. It will be appreciated that in an efficient manufacturing scenario, the handle may be a unitary piece of molded material without departure from the invention.

The handle 14 is divided into forward and aft zones, and, in order to assure an ergonomic fit with the users hand, the ribs are gently curved in the forward zone, as at 21, retreating from the cup 12 toward a transverse strap 23 which encircles the ribs at approximately half the length of the ribs and separates the forward and aft zones. The handle is gently arched moving rearwardly of the strap 23 in order to fit comfortably in the palm of the user's hand. The ergonomics of the handle and cup are enhanced by creating an angle, α as shown in FIG. 2, which angle is formed by a straight, longitudinally extending, base line 22 extending through the approximate center of the tongue cleaner, relative to a reference line L, which intersects the base line at the end of the handle to form an angle which can comfortably range from between 10° and 17°.

The cup 12 is perhaps the most important element of the tongue scraper in that it provides the user with the ability to remove bacteria and other impediments from the surface of the tongue T, and particularly the posterior region of the tongue. To accomplish this salutary objective, the cup 12 is uniquely shaped to caress the surface of the tongue, without being either too narrow or too wide, to quickly achieve its purpose.

Accordingly, the cup 12 is provided with a continuous closed side wall rising from a bottom wall 27, to form an enclosed reservoir therewith. The side wall 25 is relatively straight at the intersection with the handle 14, as at 29. From this straight section 29, the side wall is formed with divergent sections 32 which curve gently outwardly, away from the straight section 29 where they seamlessly connect at a forward or leading edge section defining a relatively straight scraping edge 34, giving the cup, as defined by the shape of the bottom wall as viewed in FIG. 1, the appearance of a truncated triangle.

The leading edge portion of the cup 34 is uniquely designed to gently remove material from the surface of the tongue only upon movement from the rear toward the front of the tongue so as to eliminate any possibility of cutting, or otherwise injuring, the tongue's surface. To this end, and with particular reference to FIG. 5, a scraping surface 36 is oriented generally transverse to, and remote from, the longitudinal axis of the handle 14 in order that a gentle movement of the handle along its longitudinal axis will result in the removal of bacteria and other impediments from the portion of the tongue engaged by the scraping edge. The scraping edge 36 is at the intersection of the terminus of the arcuate portion 38 of the leading edge surface and the interior wall 41 of the cup, and is, thus, is in the plane of, but inboard, of the leading edge to thereby protect the surface of the tongue from inadvertent abrasion which might otherwise result from use of the device.

The material, of whatever nature, removed by the gentle scraping action is accumulated in the reservoir cup 12, providing a two fold benefit, the first being stark visual evidence of the existence of foreign matter on the tongue, thereby encouraging at least daily removal thereof as a part of an oral hygiene regimen, and secondly, providing a pocket for the accumulation of such foreign matter which facilitates its convenient and sanitary disposal.

Having described a preferred embodiment of the present invention, it will be appreciated by those skilled in the art that some variation in the specific structure, although not expressly described, is well within the contemplation of the invention as described in the accompanying claims, wherein:

1. A tongue cleaner having a reservoir cup; a handle, said handle extending longitudinally outwardly from said cup and affixed thereto to permit digital manipulation thereof across the surface of a user's tongue; said cup being defined by a bottom wall encircled by a side wall, said bottom wall being generally triangular in shape, said side wall having a forward section, said forward, leading edge, section being essentially straight and transverse to the axis of said handle;
a scraping edge on said forward leading edge section such that said scraping edge moves gently over the tongue's surface as the tongue cleaner is moved from aft toward the front of the tongue.

2. The tongue cleaner of claim 1, wherein said forward, leading edge section comprises an arcuate portion, said arcuate portion terminating in a scraping edge.

3. The tongue cleaner of claim 2, wherein said scraping edge is inboard of said arcuate portion.

4. The tongue cleaner of claim 1, wherein said forward, leading edge portion is remote from the intersection of said handle and said cup.

5. The tongue cleaner of claim 2, wherein said forward, leading edge portion is remote from the intersection of said handle and said cup.

6. The tongue cleaner of claim 1, wherein said cup comprises a bottom wall, a continuous side wall encircling said bottom wall, and having divergent side wall sections adjacent and contiguous with said forward, leading edge section.

7. The tongue cleaner of claim 2, wherein said cup comprises a bottom wall, a continuous side wall encircling said bottom wall, and having divergent side wall sections adjacent and contiguous with said forward, leading edge section.

8. The tongue cleaner of claim 1, wherein said handle is curved to comfortably fit the hand of the user.

9. The tongue cleaner of claim 2, wherein said handle is curved to comfortably fit the hand of the user.

10. The tongue cleaner of claim 8, wherein said curvature of said handle forms an angle with a flat surface of between 10 and 17 degrees.

11. The tongue cleaner of claim 10, wherein said angle is 14 degrees.
